(19) United States
(12) Patent Application Publication
(10) Pub. No.: US 2010/0099115 A1
(43) Pub. Date: Apr. 22, 2010

(54) SYSTEMS AND METHODS FOR PREPARING AND ANALYZING SAMPLES

(76) Inventors: Patrick A. Mach, Shorewood, MN (US); Lynn P. Cloney, Vancouver (CA); Chunmei Guo, Woodbury, MN (US); Paul C. Harris, Bothell, WA (US); Kevin R. Katzenmaier, Woodbury, MN (US); Barbara R. Kinnaird, Richmond (CA); Mara S. Reif-Wenner, Red Wing, MN (US); Michelle L. Rosauer, North St. Paul, MN (US); Jeffrey D. Smith, Marine on St. Croix, MN (US); Heather M. Webb, Woodbury, MN (US)

Correspondence Address: 3M INNOVATIVE PROPERTIES COMPANY, PO BOX 33427, ST. PAUL, MN, 55133-3427, US

(21) Appl. No.: 12/515,789

(22) PCT Filed: Nov. 20, 2007
(86) PCT No.: PCT/US07/85242

§ 371 Date: Dec. 15, 2009

Related U.S. Application Data
(60) Provisional application No. 60/867,102, filed on Nov. 22, 2006.
(60) Provisional application No. 60/867,093, filed on Nov. 22, 2006.
(60) Provisional application No. 60/867,012, filed on Nov. 22, 2006.
(60) Provisional application No. 60/860,763, filed on Nov. 22, 2006.

(51) Int. Cl. G01N 33/53; C12M 1/34; B01L 3/00; G01N 33/569

(52) U.S. Cl. 435/7.1; 435/287.1; 422/102

(57) ABSTRACT

The invention relates to systems and methods for preparing and analyzing samples (e.g., mucosal samples) for a microorganism of interest. In particular, the systems and methods are useful for detecting one or more analytes characteristic of a microorganism (i.e., microbe) of interest, such as components of cell walls that are characteristic of a microbe, particularly Staphylococcus aureus.

### RELATED APPLICATIONS

The present application claims the benefit of U.S. Provisional Patent Application Ser. Nos. 60/867,093, 60/867,012, 60/867,102, 60/860,763, all filed on Nov. 22, 2006, and all of which are incorporated herein by reference in their entirety.

### BACKGROUND

The emergence of bacteria having resistance to commonly used antibiotics is an increasing problem with serious implications for the treatment of infected individuals. Accordingly, it is of increasing importance to determine the presence of such bacteria at an early stage and in a relatively rapid manner to gain better control over such bacteria. This also applies to a variety of other microbes.

One such microbe of significant interest is Staphylococcus aureus (“S. aureus”). This is a pathogen causing a wide spectrum of infections including: superficial lesions such as small skin abscesses and wound infections; systemic and life threatening conditions such as endocarditis, pneumonia and septicemia; as well as toxinoses such as food poisoning and toxic shock syndrome. Some strains (e.g., Methicillin-Resistant S. aureus) are resistant to all but a few select antibiotics.

Current techniques for the detection of microbes, particularly bacteria resistant to antibiotics, are generally time consuming and typically involve culturing the bacteria in pure form. One such technique for the identification of pathogenic staphylococci associated with acute infection, i.e., S. aureus in humans and animals and S. intermedius and S. hyicus in animals, is based on the microbe's ability to clot plasma. At least two different coagulase tests have been described: a tube test for free coagulase and a slide test for “cell bound coagulase” or clumping factor. The tube coagulase test typically involves mixing an overnight culture in brain heart infusion broth with reconstituted plasma, incubating the mixture for 4 hours and observing the tube for clot formation by slowly tilting the tube for clot formation. Incubation of the test overnight has been recommended for S. aureus since a small number of strains may require longer than 4 hours for clot formation. The slide coagulase test is typically faster and more economical; however, 10% to 15% of S. aureus strains may yield a negative result, which requires that the isolate be reexamined by the tube test.

Although methods of detecting S. aureus, as well as other microbes, have been described in the art, there would be advantage in improved methods of sample preparation and analysis (e.g., detection).

### SUMMARY

The invention provides systems and methods for preparing and analyzing a sample, for example, for a microorganism of interest. In particular, the systems and methods are useful for detecting one or more analytes characteristic of a microorganism (i.e., microbe) of interest, such as components of cell walls that are characteristic of a microbe, particularly Staphylococcus aureus.

In one embodiment, the present invention provides a method of analyzing a sample for a bacterium, the method comprising: providing a sample suspected of including one or more analytes characteristic of a specific bacterium; providing an analyte-binding material comprising two or more antibodies having antigenic specificities for two or more distinct analytes characteristic of the specific bacterium; providing an immunochromatographic device (preferably a lateral flow device) comprising a sample capture zone, wherein the sample capture zone comprises a mixture of two or more antibodies having antigenic specificities for two or more distinct analytes characteristic of the specific bacterium; providing contact between the sample, the analyte-binding material, and the sample capture zone of the immunochromatographic device; wherein, for each of the analytes present, at least one of the antigenic specificities of antibodies in the sample capture zone is not functionally blocked from binding to its analyte by the analyte-binding material, having a different antigenic specificity, with analyte bound thereto; and analyzing for the presence or absence of the specific bacterium.

Preferably, in the above-described method, providing contact between the sample, the analyte-binding material, and the sample capture zone of the immunochromatographic device comprises: contacting the sample with the analyte-binding material; and contacting the sample having analyte-binding material therein with the sample capture zone of the immunochromatographic device. Alternatively, providing contact between the sample, the analyte-binding material, and the sample capture zone of the immunochromatographic device comprises: placing the sample into the immunochromatographic device; and subsequently contacting the sample in the device with the analyte-binding material.

In certain embodiments, providing a sample comprises: placing a sample into a vial (i.e., tube), wherein the vial includes a first reagent, and maintaining contact between the sample and first reagent under conditions sufficient for reaction between one or more components of the sample and the first reagent; and subsequently placing a cap on the vial, wherein the cap includes a second reagent, and mixing the contents of the vial and the second reagent under conditions sufficient for reaction between one or more components in the vial and the second reagent. Preferably, before reaction, the first reagent in the vial (i.e., tube), the second reagent in the cap, or both, is in solid or semi-solid form. In certain embodiments, the cap is a first cap, and after the step of placing the first cap on the vial, the method further comprises removing the first cap, replacing it with a second cap that includes a third reagent, and mixing the contents of the vial and the third reagent under conditions sufficient for reaction between one or more components in the vial and the third reagent. In certain embodiments, the method further includes adding a diluent to the contents of the vial after removing the first cap and replacing it with the second cap.

The present invention also provides an immunochromatographic device comprising: a sample flow path; and a sample capture zone formed on or in a porous material within the sample flow path, the sample capture zone comprising an antibody selected from the group consisting of MAb-76, MAb-107, affinity-purified RxClf40, and combinations thereof. In certain embodiments, the porous material comprises a membrane (e.g., a membrane of nitrocellulose or nylon). In certain embodiments, the sample capture zone includes at least two antibodies that bind to different analytes.

The present invention also provides a system comprising: an immunochromatographic device comprising: a sample flow path; and a sample capture zone formed on or in a porous material within the sample flow path, the sample capture zone comprising one or more antibodies. The system further includes particulate material comprising one or more antibodies disposed thereon. In this system the one or more antibodies of the sample capture zone or disposed on the particulate material, or both, are selected from the group consisting of MAb-76, MAb-107, affinity-purified RxClf40, and combinations thereof. In certain embodiments, the antibodies are antibodies MAb-76 and affinity-purified RxClf40 in a 1:1 ratio disposed on particulate material (although other ratios are also possible). In certain embodiments, each particle of the particulate material has at least two antibodies that bind different analytes disposed thereon. In certain embodiments, the particulate material comprises polystyrene and/or latex beads. In certain embodiments, the sample capture zone comprises at least two antibodies that bind different analytes.

The present invention also provides a sample preparation system comprising: at least one vial and at least one cap for such vial, wherein the vial and the cap each has a different sample preparation reagent disposed therein for the sequential treatment of a sample for analysis of a target analyte; wherein at least one of the sample preparation reagents comprises an analyte-binding material comprising: a solid support material; an antibody disposed on the solid support material, wherein the antibody is selected from the group consisting of MAb-76, MAb-107, affinity-purified RxClf40, and combinations thereof; and an optional detectable marker; an immunochromatographic device comprising: a sample flow path; and a sample capture zone formed on or in a porous material within the sample flow path, the sample capture zone comprising a mixture of two or more antibodies having antigenic specificities for two or more distinct analytes characteristic of a specific bacterium; optionally, at least one sample transfer device; and optionally, at least one other sample preparation reagent not in the cap or vial.

In another embodiment, a sample preparation system includes: at least one vial and at least one cap for such vial, wherein the vial and the cap each has a different sample preparation reagent disposed therein for the sequential treatment of a sample for analysis of a target analyte; wherein at least one of the sample preparation reagents comprises an analyte-binding material comprising: a solid support material; two or more antibodies disposed on the solid support material, wherein the two or more antibodies have antigenic specificities for two or more distinct analytes characteristic of a specific bacterium; and an optional detectable marker; an immunochromatographic device comprising: a sample flow path; a sample capture zone formed on or in a porous material within the sample flow path, the sample capture zone comprising an antibody selected from the group consisting of MAb-76, MAb-107, affinity-purified RxClf40, and combinations thereof; and optionally, at least one sample transfer device; and optionally, at least one other sample preparation reagent not in the cap or vial.

The present invention also provides methods of preparing a mucosal test sample. In such methods, a mucosal sample (e.g., a nasal mucosal sample) suspected of containing a microorganism is combined with an enzymatic lysing agent. The enzymatic lysing agent can include, for example, at least one member of the group consisting of: lysostaphin, pepsin, glucosidase, galactosidase, lysozyme, achromopeptidase, endopeptidase, N-acetylmuranyl-L-alanine amidase, endo-beta-N-acethylglucosaminidase, ALE-I, and combinations thereof. In a preferred embodiment, the enzymatic lysing agent includes lysostaphin.

The mucosal test sample and enzymatic lysing agent are combined sequentially with a mucolytic agent that is distinct from the enzymatic lysing agent, thereby forming a mucosal test sample. In certain embodiments, the mucosal sample and the enzymatic lysing agent are combined, and thereafter the sample and enzymatic lysing agent are combined with the mucolytic agent. For example, the mucosal sample and the enzymatic lysing agent can be incubated for a time sufficient to allow lysis of the microorganism and release of at least some antigenic components of the microorganism, prior to combining the sample and enzymatic lysing agent with the mucolytic agent.

The mucolytic agent can include, for example, at least one member of the group consisting of: an enzyme, a salt, a reducing agent, an acid, and combinations thereof. For example, the mucolytic agent can include a reducing agent, such as beta-mercapto ethanol (BME), dithiotreotol (DTT), dithioerythritol (DTE), cysteine, tris(2-carboxyethyl) phosphine hydrochloride (TCEP; Pierce Chemical Company, Rockford, Ill.), n-acetyl cysteine, or combinations thereof. In a preferred embodiment, the reducing agent includes n-acetyl cysteine. The reducing agent can have a pH of less than 3.

In other embodiments of the invention, the mucosal test sample is combined with a surfactant or detergent subsequently to, or concurrently with, combining the sample and enzymatic lysing agent with the mucolytic agent. The surfactant can include, for example, an anionic surfactant, such as sodium-dodecyl sulfate (SDS) or sodium lauryl sulfate (SLS). The mucosal test sample and surfactant can be further subsequently combined with a neutralizing buffer, such as a buffer that is sufficient to adjust the pH of the mucolytic test sample and surfactant to a range of 5 to 8.

The methods of the present invention can further include a step of combining the mucosal test sample with an analyte recognition element, whereby the presence of an analyte characteristic of the microorganism can be detected. In certain embodiments, the microorganism includes Staphylococcus aureus. In those embodiments, the analyte recognition element can include at least one antibody that binds an antigenic component of Staphylococcus aureus that is released upon lysis consequent to combination with the enzymatic lysing agent (e.g., lysostaphin). The analyte recognition element can be labeled with a detectable marker, either directly or indirectly, thereby forming a labeled recognition element, and can also include a component that can cause conjugation of the analyte recognition element to a solid support regardless of the presence of antigen components in the mucosal test sample. In certain preferred embodiments, the analyte recognition element (and preferably, a labeled analyte recognition element) includes at least two antibodies, whereby the antibodies bind, independently, distinct antigenic components of Staphylococcus aureus produced by lysis, or bind, independently, distinct epitopes of a common antigenic component produced by the lysis. Representative antibodies that bind to antigenic components of Staphylococcus aureus include MAb-76, MAb-107, affinity-purified RxClf40, and combinations thereof.

The sample preparation methods of the invention are useful for providing the release and/or accessibility of one or more antigenic components characteristic of a microorganism (i.e., microbe) of interest. The methods ensure not only release of antigenic components from within a microbe, such as components of cell walls that are characteristic of a microbe, but also disintegration of mucus, enhancing the availability of antigenic components for targeting with analyte recognition elements, particularly labeled recognition elements (e.g., labeled antibodies) and reducing the viscosity of the mixture allowing the antigenic components and the analyte recognition elements, particularly labeled recognition elements (e.g., labeled antibodies) to move more freely and thus combine.

In a preferred embodiment, the present invention provides a method for preparing a nasal mucosal test sample, comprising the steps of combining a nasal mucosal sample of a Staphylococcus aureus with lysostaphin; subsequently combining the nasal mucosal sample and lysostaphin with n-acetyl cysteine having a pH below 3 and with sodium dodecyl sulfate; inactivating the combined nasal mucosal sample, lysostaphin, n-acetyl cysteine and sodium dodecyl sulfate by neutralizing the pH to a range of 5 to 8 to thereby form a neutralized test sample; and combining the neutralized test sample with a labeled recognition element that includes at least two antibodies selected from the group consisting of MAb-76, MAb-107, RXCLF 40, and combinations thereof. In such method, preferably, the nasal mucosal sample and lysostaphin are incubated for a time sufficient to allow lysis of the Staphylococcus aureus and release of at least some of its antigenic components.

The present invention also provides systems and methods for sample preparation. For example, in one embodiment, a sample preparation system includes: at least one vial (e.g., tube) and at least one cap for such vial, wherein the vial and the cap each has a different sample preparation reagent disposed therein for the sequential treatment of a sample for analysis of a target analyte; optionally, at least one sample transfer device; and optionally, at least one other sample preparation reagent not in the cap or vial.

In another embodiment, a sample preparation system includes: at least one vial and two or more caps for such vial, wherein each of the caps has a different sample preparation reagent disposed therein for the sequential treatment of a sample for analysis of a target analyte; optionally, at least one sample transfer device; and optionally, at least one other sample preparation reagent not in the caps.

Such sample preparation systems could be used in the following exemplary manner: placing a sample into a vial, wherein the vial includes a first reagent, and maintaining contact between the sample and first reagent under conditions sufficient for reaction between one or more components of the sample and the first reagent; and subsequently placing a cap on the vial, wherein the cap includes a second reagent, and mixing the contents of the vial and the second reagent under conditions sufficient for reaction between one or more components in the vial and the second reagent. The first reagent in the vial, the second reagent in the cap, or both (before reaction) can be in solid or semi-solid form (e.g., in a dried-down coated or spotted form). In one embodiment, the cap referenced above can be a first cap, and after the step of placing the first cap on the vial, the method can further include removing the first cap, replacing it with a second cap that includes a third reagent, and mixing the contents of the vial and the third reagent under conditions sufficient for reaction between one or more components in the vial and the third reagent.

Another option is where there is no reagent in the vial and that all reagents are added by the addition of caps containing reagents.

In another embodiment, the present invention provides a method of sample preparation comprising: providing at least one vial and two or more caps for such vial, wherein each of the caps has a different sample preparation reagent disposed therein for the sequential treatment of a sample for analysis of a target analyte; placing a sample into a vial, placing each cap on the vial in a desired sequence, and mixing the contents of the vial and each cap under conditions sufficient for reaction between one or more components in the vial and each sample preparation reagent.

### DEFINITIONS

The term “immunochromatographic device” herein refers to a non-enzymatic detection assay device containing a porous material (preferably in the form of a membrane (e.g., a multi-layered material)) that allows for fluid flow through the material. The non-enzymatic detection does not involve a structural conformation change of the detectable marker (e.g., fluorescent tags or labels).

The terms “inactivate” or “inactivating” or “inactivation” refer to stopping the activity of a reagent or stopping a reaction, for example, which can occur by a wide variety of mechanisms, including, for example, blocking, diluting, inhibiting, denaturing, competing, etc.

The terms “analyte” and “antigen” are used interchangeably and refer to various molecules (e.g., Protein A) or epitopes of molecules (e.g., different binding sites of Protein A), or whole cells of the microorganism, that are characteristic of a microorganism (i.e., microbe) of interest. These include components of cell walls (e.g., cell-wall proteins such as protein A, and Clumping Factor, which is a cell wall-associated fibrinogen receptor that is found in S. aureus), external cell components (e.g., capsular polysaccharides and cell-wall carbohydrates), internal cell components (e.g., cytoplasmic membrane proteins), etc.

The term “mucus-containing sample” or “mucosal test sample” refers to samples that include, or are derived from, mucosal membranes and mucosal tissues, which are used interchangeably and refer to the surfaces of the nasal (including anterior nares, nasopharyngeal cavity, etc.), oral (e.g., mouth), outer ear, middle ear, vaginal cavities, and other similar tissues. Examples include mucosal membranes such as buccal, gingival, nasal, ocular, tracheal, bronchial, gastrointestinal, rectal, urethral, ureteral, vaginal, cervical, and uterine mucosal membranes.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

As used herein, “a,” “an,” “the,” “at least one,” and “one or more” are used interchangeably. Thus, for example, an analyte-binding material that comprises “an” antibody can be interpreted to mean that the analyte-binding material includes “one or more” antibodies that bind different analytes.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 is a schematic of an exemplary sample preparation system and method using caps and vials having reagents therein.

### DETAILED DESCRIPTION OF ILLUSTRATIVE EMBODIMENTS

The present invention is directed to various systems and methods of preparing a sample, particularly for analyzing a sample for a microorganism (i.e., microbe) of interest based on analysis of one or more analytes characteristic of the microorganism of interest. The systems and methods of the present invention can involve not only detecting the presence of an analyte characteristic of the microorganism of interest, but preferably identifying such analyte, which can lead to identifying a microbe for which the analyte is characteristic. In certain embodiments, analyzing the sample includes quantifying the analyte characteristic of the microorganism of interest.

Preferably, methods of the present invention include the use of a first set of two or more antibodies having antigenic specificities for two or more distinct analytes characteristic of the specific microorganism (preferably, a bacterium), the use of a second set of two or more antibodies having antigenic specificities for two or more distinct analytes characteristic of the specific microorganism, wherein, for each of the analytes present, at least one of the antigenic specificities of antibodies in the second set is not functionally blocked from binding to its analyte by the first set of antibodies, having a different antigenic specificity, with analyte bound thereto. Such antibodies are preferably cooperative in their binding characteristics. That is, they are capable of simultaneously binding to distinct regions of the target analyte(s) or optimally are found to be of complementary binding whereby the binding of their distinct analytes by one antibody set is enhanced by the binding of one or more other antibody sets.

Microbes (i.e., microorganisms) of particular interest include prokaryotic and eukaryotic organisms, particularly Gram positive bacteria, Gram negative bacteria, fungi, protozoa, mycoplasma, yeast, viruses, and even lipid-enveloped viruses. Particularly relevant organisms include members of the family Enterobacteriaceae, or the family Micrococcaceae or the genera Staphylococcus spp., Streptococcus spp., Pseudomonas spp., Enterococcus spp., Salmonella spp., Legionella spp., Shigella spp. Yersinia spp., Enterobacter spp., Escherichia spp., Bacillus spp., Listeria spp., Vibrio spp., Corynebacteria spp. as well as herpes virus, Aspergillus spp., Fusarium spp., and Candida spp. Particularly virulent organisms include Staphylococcus aureus (including resistant strains such as Methicillin Resistant Staphylococcus aureus (MRSA)), S. epidermidis, Streptococcus pneumoniae, S. agalactiae, S. pyogenes, Enterococcus faecalis, Vancomycin Resistant Enterococcus (VRE), Vancomycin Resistant Staphylococcus aureus (VRSA), Vancomycin Intermediate-resistant Staphylococcus aureus (VISA), Bacillus anthracis, Pseudomonas aeruginosa, Escherichia coli, Aspergillus niger, A. fumigatus, A. clavatus, Fusarium solani, F. oxysporum, F. chlamydosporum, Listeria monocytogenes, Listeria ivanovii, Vibrio cholera, V. parahemolyticus, Salmonella cholerasuis, S. typhi, S. typhimurium, Candida albicans, C. glabrata, C. krusei, Enterobacter sakazakii, E. coli O157 and multiple drug resistant Gram negative rods (MDR).

Gram positive and Gram negative bacteria are of particular interest. Of even more interest are Gram positive bacteria, such as Staphylococcus aureus. Typically, these can be detected by detecting the presence of a cell-wall component characteristic of the bacteria, such as a cell-wall protein. Also, of particular interest are antibiotic resistant microbes including MRSA, VRSA, VISA, VRE, and MDR. Typically, these can be detected by additionally detecting the presence of an internal cell component, such as a membrane protein, transport protein, enzyme, etc., responsible for antibiotic resistance.

The present invention is advantageous over conventional techniques for analyzing samples for such microbes because the signal for the analyte (e.g., external cell-associated components and/or cell markers from the internal portion of the cells) characteristic of the microbe is enhanced. Methods of the present invention could be used to analyze a sample for separate molecules (e.g., molecules like protein A and Clumping Factor for analysis of Staphylococcus aureus) or two different epitopes of the same molecule (e.g., a protein).

Such analytes include, for example, cell-wall proteins such as protein A and microbial surface components recognizing adhesive matrix molecules (MSCRAMMs) such as fibrinogen-binding proteins (e.g., clumping factors), fibronectin-binding proteins, collagen-binding proteins, heparin-related polysaccharides binding proteins, and the like. Protein A and clumping factors, such as fibrinogen-binding factors and clumping factors A, B, and Efb, are also particularly useful in methods of detecting the presence of Staphylococcus aureus. Other cell-wall components of interest include capsular polysaccharides and cell-wall carbohydrates (e.g., teichoic acid and lipoteichoic acid).

If desired, methods of the present invention can further include analyzing the sample for an internal cell component, which may or may not be associated with a cell membrane, as the analyte of interest. Internal cell components are particularly useful in analyzing antibiotic resistant microbes, such as MRSA, VRSA, VISA, VRE, and MDR. Internal cell components that can be characteristic of such microbes include membrane proteins. Examples of such membrane proteins include cytoplasmic membrane proteins, outer membrane proteins, and cell membrane proteins. Cytoplasmic membrane proteins, such as penicillin binding proteins (PBP) (e.g., PBP2′ or PBP2a) can be particularly characteristic of antibiotic resistant microbes. For example, the cytoplasmic membrane protein PBP2′ is characteristic of MRSA.

Species of interest can be analyzed in a test sample that may be derived from a wide variety of sources, such as a physiological fluid, e.g., blood, saliva, ocular lens fluid, synovial fluid, cerebral spinal fluid, pus, sweat, exudate, urine, mucus, lactation milk, or the like. Further, the test sample may be derived from a body site, e.g., wound, skin, nares, scalp, nails, etc.

Samples of particular interest include mucus-containing samples, such as nasal samples (from, e.g., anterior nares, nasopharyngeal cavity, nasal cavities, anterior nasal vestibule, etc.), as well as samples from the outer ear, middle ear, mouth, rectum, vagina, or other similar tissue. Examples of specific mucosal tissues include buccal, gingival, nasal, ocular, tracheal, bronchial, gastrointestinal, rectal, urethral, ureteral, vaginal, cervical, and uterine mucosal membranes.

Besides physiological fluids, other test samples may include other liquids as well as solid(s) dissolved in a liquid medium. Samples of interest may include process streams, water, soil, plants or other vegetation, air, surfaces (e.g., contaminated surfaces), and the like.

The art describes various patient sampling techniques for the detection of microbes, such as S. aureus. Such sampling techniques are suitable for the methods of the present invention as well. For example, it is common to obtain a sample by wiping the nares of a patient. A particularly preferred sampling technique includes swabbing the subject's (e.g., patient's) anterior nares with a sterile swab or sampling device. For example, one swab is used to sample each subject, i.e., one swab for both nares. The sampling can be performed, for example, by inserting the swab dry or pre-moistened with an appropriate solution into the anterior tip of the subject's nares and rotating the swab for two complete revolutions along the nares' mucosal surface.

A wide variety of swabs or other sample collection devices are commercially available, for example, from Puritan Medical Products Co. LLC, Guilford, Me., under the trade designation PURE-WRAPS, or from Copan Diagnostics, Inc., Murrietta, Calif., under the trade designations microRheologics nylon flocked swab and ESwab Collection and Transport System. A sample collection means such as that disclosed, for example, in U.S. Pat. No. 5,879,635 (Nason) can also be used if desired. Swabs can be of a variety of materials including cotton, rayon, calcium alginate, Dacron, polyester, nylon, polyurethane, and the like.

The sample collection device (e.g., swab) can then be cultured directly, analyzed directly, or extracted with an appropriate solution. Such extraction (i.e., elution) solutions typically including water and can optionally include a buffer and at least one surfactant. An example of an elution buffer includes, for example, phosphate buffered saline (PBS) with TWEEN 20 or with PLURONIC L-64. Other extraction solutions function to maintain specimen stability during transport from sample collection site to sample analysis sites. Examples of these types of extraction solutions include Amies' and Stuart's transport media.

The test sample (e.g., liquid) may be subjected to treatment prior to further analysis. This includes concentration, precipitation, filtration, centrifugation, distillation, dialysis, dilution, inactivation of natural components, addition of reagents, chemical treatment, etc.

That is, the test sample can be prepared using a wide variety of means well-known to those of skill in the art. For example, the sample could be disrupted to make available for analysis an analyte characteristic of the specific microorganism of interest using physical means (e.g., sonication, pressure, boiling or other heating means, vortexing with glass beads, etc.). Alternatively, the sample could be disrupted to make available for analysis an analyte characteristic of the specific microorganism of interest using various chemical reagents, which can include one or more components.

In certain embodiments, methods of the present invention include lysing the cells in the test sample. In the methods of the present invention, lysing can include contacting the cells with a lysing agent or physically lysing the cells. Lysing can be conducted under conventional conditions, such as, for example, at a temperature of 5° C. to 42° C. (probably as high as 50° C.), preferably at a temperature of 15° C. to 25° C. Significantly, the lysing can occur using uncultured cells, i.e., a direct test sample, although cultured cells can be used as well.

Lysing can occur upon physically lysing the cells. Physical lysing can occur upon vortexing the test sample with glass beads, sonicating, heating and boiling, or subjecting the test sample to high pressure, such as occurs upon using a French press, for example.

Lysing can also occur using a lysing agent. Suitable lysing agents include, for example, enzymes (e.g., proteases, glycosidases, nucleases). Exemplary enzymes for lysing (i.e., enzymatic lysing agents) include lysostaphin, pepsin, glucosidase, galactosidase, lysozyme, achromopeptidase, endopeptidases, N-acetylmuramyl-L-alanine amidase, endo-beta-N-acethylglucosaminidase, ALE-1, DNase, and RNase. Various combinations of enzymes can be used if desired. A preferred enzyme is selected from the group consisting of lysostaphin, pepsin, glucosidase, galactosidase, lysozyme, achromopeptidase, endopeptidases, N-acetylmuramyl-L-alanine amidase, endo-beta-N-acethylglucosaminidase, ALE-1, and combinations thereof. Lysostaphin is particularly useful in methods of detecting the presence of Staphylococcus aureus.

Other lysing agents include salts (e.g., chaotrophic salts), solubilizing agents (e.g., detergents), reducing agents (e.g., beta-mercaptoethanol (BME), dithiothreitol (DTT), dithioerythritol (DTE), cysteine, TCEP, n-acetyl cysteine)), acids (e.g., HCl), and bases (e.g., NaOH). Such lysing agents may be more suitable for certain organisms than for others, for example, they can be more suitable for use with Gram negative bacteria than with Gram positive bacteria.

Various combinations of such lysing agents and methods can be used if desired.

Methods of lysing are further discussed in U.S. Pat. App. Pub. No. 2005/0153370 A1. In particular, such lysing methods involve detecting one or more components of cell walls that are characteristic of a species of interest (e.g., a microbe of interest), and optionally, one or more internal cell components that are further characteristic of a species of interest (e.g., an antibiotic resistant microbe of interest). It is believed that the cell-wall fragments analyzed are solid pieces of cell wall. That is, it is believed that they are not solubilized upon lysing; rather, the cell wall is merely broken into pieces. Furthermore, the cell-wall component that is analyzed is still part of (i.e., in or on) the cell wall fragments. That is, they are not solubilized upon lysing. Significantly, this enhances the signal of the cell-wall component relative to the same component in an unlysed cell.

Additionally, if desired, and the sample is a mucus-containing sample, it can be further treated, either before or after lysing, with at least one reagent that can include a mucolytic agent. Treatment of mucus-containing samples with mucolytic agents can reduce the interference resulting from the presence of mucus during the analysis.

Examples of mucolytic agents include enzymes (e.g., pepsin, DNases, RNases, glucosidases, galactosidases, glycosidases), salts (e.g., chaotrophic salts), solubilizing agents (e.g., surfactants, detergents), reducing agents (e.g., beta-mercapto ethanol (BME), dithiotreotol (DTT), dithioerythritol (DTE), cysteine, TCEP, n-acetyl cysteine), and acids (e.g., HCl). Various combinations of such mucolytic agents can be used if desired. One of skill in the art will understand that there can be overlap between lysing agents and mucolytic agents; although not all lysing agents will be mucolytic, for example.

In certain embodiments, a mucosal sample and an enzymatic lysing agent are combined for a time sufficient to allow lysis of the microorganism and release of at least some antigenic components of the microorganism; subsequently, the sample and enzymatic lysing agent are combined with a mucolytic agent that is distinct from the enzymatic lysing agent.

In a preferred embodiment, if the sample is a mucus-containing sample, after lysing, the sample can be contacted with a first reagent under conditions sufficient for reaction between one or more components of the mucus-containing sample and the first reagent to form a composition. In such an embodiment, the first reagent can include one or more reducing agents, preferably acidified (e.g., having a pH of less than 3). Examples of such reducing agents include beta-mercapto ethanol (BME), dithiothreitol (DTT), dithioerythritol (DTE), cysteine, TCEP, and n-acetyl cysteine. A preferred reducing agent is n-acetyl cysteine, which is preferred because it is relatively stable and can be oxidized easily. Reducing agents can be acidified using a variety of acids, such as inorganic acids (e.g., HCl) or organic acids (e.g., lactic acid, citric acid). Alternatively, if used in sufficiently high concentrations, the pH of the reducing agent does not need to be adjusted with an acid. Also, alternatively, an acid alone (e.g., HCl) can be used as the mucolytic agent.

Typically, but optionally, after adding a reducing agent, the sample preparation involves inactivating the reducing agent in the composition. This can be done, for example, by providing a competitive substrate (for example, bovine serum albumen for n-acetyl cysteine). Other examples of reagents that inactivate the reducing agent include a diluent including a neutralizing buffer. Representative ingredients for neutralizing buffers can include, for example, buffering agent(s) (e.g., phosphate), salt(s) (e.g., NaCl), protein stabilizer(s) (e.g., bovine serum albumin (BSA), casein, serum) polymer(s), saccharides, and/or detergent(s) or surfactant(s) (e.g., one or more of the following agents listed by tradenames and commonly available sources: NINATE 411 (amine alkylbenzene sulfonate, available from Stepan Co., Northfield, Ill.), ZONYL FSN 100 (Telomer B monoether with polyethylene glycol, available from E.I. DuPont de Nemours Co.), Aerosol OT 100% (sodium dioctylsulfosuccinate, available from American Cyanamide Co.), GEROPON T-77 (sodium N-oleyl-N-methyltaurate, available from Rhodia Novacare), BIO-TERGE AS-40 (sodium olefin (C₁₄-C₁₆)sulfonate, available from Stepan Co.), STANDAPOL ES-1 (sodium polyoxyethylene(1) laurylsulfate, available from Cognis Corp., Ambler, Pa.), TETRONIC 1307 (ethylenediamine alkoxylate block copolymer, available from BASF Corp.), SURFYNOL 465, 485, and 104 PG-50 (all available from Air Products and Chemicals, Inc.), IGEPAL CA210 (octylphenol ethoxylate, available from Stepan Co.), TRITON X-45, X-100, and X-305 (octylphenoxypolyethoxy ethanols, all available from The Dow Chemical Co.), SILWET L-7600 (polydimethylsiloxane methylethoxylate, available from Momentive Performance Materials, Inc., Wilton, Conn.), RHODASURF ON-870 (polyethoxylated(2) oleyl alcohol, available from Rhodia Novacare), CREMOPHOR EL (polyethyoxylated castor oil, available from BASF Corp.), TWEEN 20 and TWEEN 80 (polyoxyethylene sorbitan monolaurate and monooleate, both available from Sigma-Aldrich Corp.), BRIJ 35 (polyoxyethylene(23) dodecyl ether, available from Sigma-Aldrich Corp.), CHEMAL LA-9 (polyoxyethylene(9) lauryl alcohol, available from PCC Chemax, Piedmont, S.C.), PLURONIC L64 (poly(oxyethylene-co-oxypropylene) block copolymer, available from BASF Corp.), SURFACTANT 10G (p-nonylphenoxypoly(glycidol), available from Arch Chemicals Inc., Norwalk, Conn.), SPAN 60 (sorbitan monostearate, available from Sigma-Aldrich Corp.), CREMOPHOR EL (a polyethoxylated castor oil, available from Sigma-Aldrich Corp.). If desired, the neutralizing buffer can also be used to adjust the pH of the sample.

Examples of neutralizing buffers are those disclosed in U.S. Pat. App. Pub. No. 2003/0199004, referred to as the myoglobin diluent (122 mM phosphate buffer, 100 mM NaCl, 3.3% BSA, 1.1% CREMOPHOR EL, 0.05% ProClin 300 preservative (available from Sigma-Aldrich Corp.), pH 7.2), the anthrax diluent (138 mM phosphate buffer, 138 mM NaCl, 3.6% BSA, 0.84% Surfactant 10G, 0.6% casein, 0.05% high viscosity methyl cellulose, 0.05% ProClin 300, pH 7.2), and the CKMB diluent (115 mM phosphate buffer, 115 mM NaCl, 2.3% BSA, 0.25% SURFYNOL 104 PG-50, 0.3% casein, 40 mM phenylalanine, 0.5% sheep serum, 0.05% ProClin 300 preservative, pH 7.2), and the TnI diluent (124 mM phosphate, 124 mM NaCl, 3.24% BSA, 0.76% Surfactant 10G, 0.54% casein, 405 mM GHCl, 40 mM phenylalanine, 10% goat serum, pH 7.2). A preferred diluent includes Staph A diluent (138 mM phosphate buffer, 138 mM NaCl, 3.5% BSA, 0.82% Surfactant 10G, 0.92% casein, 0.6% polyvinyl pyrrolidone of 90,000 Molecule (PVP-k90), 0.1% v/v ProClin 300 preservative, 0.1% v/v ProClin 950 preservative, pH 7.2)

In addition to, or alternative to, a reducing agent, the sample preparation of a mucus-containing sample can include the use of one or more surfactants or detergents (e.g., subsequently to or concurrently with, the combining of the sample and the enzymatic lysing agent with the mucolytic agent).

For example, a reagent that includes an acidified reducing agent can be combined with a sample and the resultant composition can be contacted with a surfactant. Alternatively, the reagent comprising the acidified reducing agent can also include a surfactant.

Suitable surfactants can be nonionic, anionic, cationic, or zwitterionic. Suitable examples include sodium dodecyl sulfate (SDS) and sodium lauryl sulfate (SLS). Preferably, the surfactant is an anionic surfactant. More preferably, the surfactant is SDS and/or SLS. Various combinations of surfactants can be used, if desired.

Optionally, the sample preparation method can include subsequently inactivating the surfactant. This can be done, for example, by providing a competitive substrate.

Other examples of inactivating the surfactant include using reagent neutralizing buffers, such as a buffer that is sufficient to adjust the pH of the mucolytic test sample and surfactant to a pH of at least 5. Preferably, the buffer is sufficient to adjust the pH to no greater than 8.

Furthermore, if one or more of the sample preparation reagents is acidic, the subsequent composition including the analyte of interest is preferably neutralized to a pH of 7 to 7.5 or near 7.2. This can be done, for example, by providing a buffer and/or a diluent of the type described above. If the diluent is used, the inactivating and neutralizing steps can occur substantially simultaneously (e.g., upon the addition of the same reagent).

Optionally, inactivating the surfactant can be done substantially simultaneously with inactivating the reducing agent and/or neutralizing the resultant composition. This can be done using, for example, buffers and/or diluents of the type described above.

In additional embodiments of the invention, the method can further include a step of combining the mucosal test sample with a labeled recognition element, such that the presence of an analyte (such as the microorganism or an antigenic component of the microorganism) can be detected, and preferably quantitatively analyzed. Representative labeled recognition elements can comprise reactant molecules for analyte binding (e.g., an analyte-binding material that includes a microorganism-recognizing reagent such as a bacteria-recognizing reagent). Such reactant molecules include antibodies, lectins, enzymes, and receptors and other binding pair technologies, as well as other reactant molecules that recognize metabolic by-products (e.g., pH changes, detectable enzyme production). For example, in one embodiment, the sample can be contacted with one or more antibodies. Such antibodies can be attached to particulate material, a membrane, or other solid support material. In some embodiments, one or more antibodies, such as an S. aureus antibody, are employed as a S. aureus reactant. “S. aureus antibody” refers to an immunoglobulin having the capacity to specifically bind a given antigen inclusive of antigen binding fragments thereof.

As mentioned above, the target analytes (i.e., analytes or components of interest) can be detected by a reactant molecule (e.g., an S. aureus reactant molecule or a bacteria-recognizing reagent for S. aureus). In some embodiments, one or more antibodies, such as an S. aureus antibody, are employed as an S. aureus reactant. “S. aureus antibody” refers to an immunoglobulin having the capacity to specifically bind a given antigen inclusive of antigen binding fragments thereof. The term “antibody” is intended to include whole antibodies of a wide variety of isotypes (e.g., IgG, IgA, IgM, IgE, etc.), and fragments thereof from vertebrate, e.g., mammalian species which are also specifically reactive with foreign compounds, e.g., proteins.

The antibodies can be monoclonal, polyclonal, or combinations thereof. Antibodies can be fragmented using conventional techniques and the fragments screened for utility in the same manner as whole antibodies. Thus, the term includes segments of proteolytically cleaved or recombinantly prepared portions of an antibody molecule that are capable of selectively reacting with a certain protein. Non-limiting examples of such proteolytic and/or recombinant fragments include Fab, F(ab′)₂, Fv, and single chain antibodies (scFv) containing a VL and/or VH domain joined by a peptide linker. The scFv's can be covalently or non-covalently linked to form antibodies having two or more binding sites. Antibodies can be labeled with a wide variety of detectable markers (i.e., detectable moieties) known to one skilled in the art. In some aspects, the antibody that binds to an analyte one wishes to measure (the primary antibody) is not labeled, but is instead detected indirectly by binding of a labeled secondary antibody or other reagent that specifically binds to the primary antibody.

In certain preferred embodiment, methods of the present invention utilize a set of two or more antibodies having antigenic specificities for two or more distinct antigenic components, or for distinct epitopes of a common antigenic component, characteristic of the microorganism, wherein, for each of the antigenic components (or epitopes of the antigenic component), at least one of the antigenic specificities of antibodies is not functionally blocked from binding to its analyte by the first set of antibodies having a different antigenic specificity, with analyte bound thereto. Such antibodies are preferably cooperative in their binding characteristics. That is, they are capable of simultaneously binding to distinct regions or epitopes of antigenic components the target analyte(s) or optimally are found to be of complementary binding whereby the binding of their distinct antigens by one antibody set is enhanced by the binding of one or more other antibodies.

In certain embodiments, methods utilize at least one antibody that binds to an antigenic component of S. aureus that is released upon lysis consequent to the combination of, for example, a mucosal sample with an enzymatic lysing agent (e.g., lysostaphin).

Various S. aureus antibodies are known in the art. For example, S. aureus antibodies are commercially available from Sigma-Aldrich and Accurate Chemical. Further, other S. aureus antibodies, such as the monoclonal antibody Mab 12-9, are described in U.S. Pat. No. 6,979,446. In certain preferred embodiments, an antibody is selected from those described herein (e.g., selected from the group consisting of MAb-76, MAb-107, affinity-purified RxClf40, affinity-purified GxClf40, MAb 12-9), fragments thereof, and combinations thereof. Such antibodies are also disclosed in U.S. patent application Ser. No. 11/562,759, filed on Nov. 22, 2006, and PCT App. Ser. No. U.S. Ser. No. 07/84,736, both entitled “ANTIBODY WITH PROTEIN A SELECTIVITY,” and in U.S. patent application Ser. No. 11/562,747, filed on Nov. 22, 2006, and PCT App. Ser. No. U.S. Ser. No. 07/84,739, both entitled “ANTIBODY WITH PROTEIN A SELECTIVITY,” and in U.S. Pat. App. Ser. No. 60/867,089, filed on Nov. 22, 2006 and U.S. patent application Ser. No. ______ (Attorney Docket No. 62611US005), filed on even date herewith, both of which are entitled “SPECIFIC ANTIBODY SELECTION BY SELECTIVE ELUTION CONDITIONS.”

Preferred antibodies are monoclonal antibodies. Particularly preferred are monoclonal antibodies that bind to Protein A of Staphylococcus aureus (also referred to herein as “S. aureus” or “Staph A”).

More particularly, in one embodiment suitable monoclonal antibodies, and antigen binding fragments thereof, are those that demonstrate immunological binding characteristics of monoclonal antibody 76 as produced by hybridoma cell line 358A76.1. Murine monoclonal antibody 76 is a murine IgG2A, kappa antibody isolated from a mouse immunized with Protein A. In accordance with the Budapest Treaty, hybridoma 358A76.1, which produces monoclonal antibody 76, was deposited on Oct. 18, 2006 in the American Type Culture Collection (ATCC) Depository, 10801 University Boulevard, Manassas, Va. 20110-2209, and was given Patent Deposit Designation PTA-7938 (also referred to herein as accession number PTA-7938). The hybridoma 358A76.1 produces an antibody referred to herein as “Mab 76.” Mab 76 is also referred to herein as “Mab76,” “Mab-76,” “MAb-76,” “monoclonal 76,” “monoclonal antibody 76,” “76,” “M76,” or “M 76,” and all are used interchangeably herein to refer to immunoglobulin produced by hybridoma cell line 358A76.1 as deposited with the American Type Culture Collection (ATCC) on Oct. 18, 2006, and assigned Accession No. PTA-7938.

In another embodiment, suitable monoclonal antibodies, and antigen binding fragments thereof, are those that demonstrate immunological binding characteristics of monoclonal antibody 107 as produced by hybridoma cell line 358A107.2. Murine monoclonal antibody 107 is a murine IgG2A, kappa antibody isolated from a mouse immunized with Protein A. In accordance with the Budapest Treaty, hybridoma 358A107.2, which produces monoclonal antibody 107, was deposited on Oct. 18, 2006 in the American Type Culture Collection (ATCC) Depository, 10801 University Boulevard, Manassas, Va. 20110-2209, and was given Patent Deposit Designation PTA-7937 (also referred to herein as accession number PTA-7937). The hybridoma 358A107.2 produces an antibody referred to herein as “Mab 107.” Mab 107 is also referred to herein as “Mab107,” “Mab-107,” “MAb-107,” “monoclonal 107,” “monoclonal antibody 107,” “107,” “M107,” or “M 107,” and all are used interchangeably herein to refer to immunoglobulin produced by the hybridoma cell line as deposited with the American Type Culture Collection (ATCC) on Oct. 18, 2006, and given Accession No. PTA-7937.

Suitable monoclonal antibodies are also those that inhibit the binding of monoclonal antibody MAb-76 to Protein A of S. aureus. The present invention can utilize monoclonal antibodies that bind to the same epitope of Protein A of S. aureus that is recognized by monoclonal antibody MAb-76. Methods for determining if a monoclonal antibody inhibits the binding of monoclonal antibody MAb-76 to Protein A of S. aureus and determining if a monoclonal antibody binds to the same epitope of Protein A of S. aureus that is recognized by monoclonal antibody MAb-76 are well known to those skilled in the art of immunology.

Suitable monoclonal antibodies are also those that inhibit the binding of monoclonal antibody MAb-107 to Protein A of S. aureus. The present invention can utilize monoclonal antibodies that bind to the same epitope of Protein A of S. aureus that is recognized by monoclonal antibody MAb-107. Methods for determining if a monoclonal antibody inhibits the binding of monoclonal antibody MAb-107 to Protein A of S. aureus and determining if a monoclonal antibody binds to the same epitope of Protein A of S. aureus that is recognized by monoclonal antibody MAb-107 are well known to those skilled in the art of immunology.

Suitable monoclonal antibodies are those produced by progeny or derivatives of this hybridoma and monoclonal antibodies produced by equivalent or similar hybridomas.

Also useful in the present invention are various antibody fragments, also referred to as antigen binding fragments, which include only a portion of an intact antibody, generally including an antigen binding site of the intact antibody and thus retaining the ability to bind antigen. Examples of antibody fragments include, for example, Fab, Fab′, Fd, Fd′, Fv, dAB, and F(ab′)₂ fragments produced by proteolytic digestion and/or reducing disulfide bridges and fragments produced from an Fab expression library. Such antibody fragments can be generated by techniques well known in the art.

Monoclonal antibodies useful in the present invention include, but are not limited to, humanized antibodies, chimeric antibodies, single chain antibodies, single-chain Fvs (scFv), disulfide-linked Fvs (sdFv), Fab fragments, F(ab′) fragments, F(ab′)₂ fragments, Fv fragments, diabodies, linear antibodies fragments produced by a Fab expression library, fragments including either a VL or VH domain, intracellularly-made antibodies (i.e., intrabodies), and antigen-binding antibody fragments thereof.

Monoclonal antibodies useful in the present invention may be of a wide variety of isotypes. The monoclonal antibodies useful in the present invention may be, for example, murine IgM, IgG1, IgG2a, IgG2b, IgG3, IgA, IgD, or IgE. The monoclonal antibodies useful in the present invention may be, for example, human IgM, IgG1, IgG2, IgG3, IgG4, IgA1, IgA2, IgD, or IgE. In some embodiments, the monoclonal antibody may be murine IgG2a, IgG1, or IgG3. With the present invention, a given heavy chain may be paired with a light chain of either the kappa or the lambda form.

Monoclonal antibodies useful in the present invention can be produced by an animal (including, but not limited to, human, mouse, rat, rabbit, hamster, goat, horse, chicken, or turkey), chemically synthesized, or recombinantly expressed. Monoclonal antibodies useful in the present invention can be purified by a wide variety of methods known in the art for purification of an immunoglobulin molecule, for example, by chromatography (e.g., ion exchange, affinity, and sizing column chromatography), centrifugation, differential solubility, or by other standard techniques for the purification of proteins.

Suitable antibodies also include a high avidity anti-Staphylococcus aureus clumping factor protein polyclonal antibody preparation that detects recombinant clumping factor (rClf40) protein of S. aureus at a concentration of preferably at least 1 picogram per milliliter (pg/mL), and more preferably up to 100 pg/mL. Suitable antibodies also include a high avidity anti-Staphylococcus aureus clumping factor protein polyclonal antibody preparation demonstrates at least a 4-fold increase in detection sensitivity in comparison to a Staphylococcus aureus clumping factor protein antiserum.

In certain embodiments, a high avidity anti-Staphylococcus aureus clumping factor protein polyclonal antibody preparation is useful, wherein the high avidity anti-S. aureus clumping factor protein polyclonal antibody preparation is prepared by a method that includes obtaining antiserum from an animal immunized with recombinant clumping factor (rClf40) protein of S. aureus; binding the antiserum to a S. aureus clumping factor (Clf40) protein affinity column; washing the column with a wash buffer having 0.5 M salt and a pH of 4; and eluting the high avidity anti-S. aureus clumping factor protein polyclonal antibody preparation from the column with an elution buffer with a pH of 2. In some embodiments, the high avidity anti-Staphylococcus aureus clumping factor protein polyclonal antibody preparation may be obtained by a method that further includes enriching the antiserum for the IgG class of antibodies prior to binding the antiserum to a S. aureus clumping factor (Clf40) protein affinity column. Such enrichment may eliminate non-immunoglobulin proteins from the preparation and/or enrich for the IgG class of antibodies within the sample.

As used herein, antiserum refers to the blood from an immunized host animal from which the clotting proteins and red blood cells (RBCs) have been removed. An antiserum to a target antigen may be obtained by immunizing a wide variety of host animals. A wide variety of immunization protocols may be used.

Antibody avidity is a measure of the functional affinity of a preparation of polyclonal antibodies. Avidity is the compound affinity of multiple antibody/antigen interactions. That is, avidity is the apparent affinity of antigen/antibody binding, not the true affinity. Despite the heterogeneity of affinities in most antisera, one can characterize such populations by defining an average affinity (K₀).

Analyte-binding material useful in methods of the present invention typically includes a solid support material. Solid support materials can include particulate materials, membranes, gels (e.g., agarose), or other solid support materials. Exemplary solid supports can include materials such as nitrocellulose, polystyrene, polypropylene, nylon, gold sols, and/or latex particles, and the like. For certain embodiments, particulate material and membranes are preferred. Preferably, for certain embodiments, the analyte-binding material is particulate material (e.g., polystyrene and/or latex beads having an average particle size of less than 1 micron, and preferably, approximately 0.3 micron).

Typically, within an immunochromatographic device is a sample flow path, and within the sample flow path is a sample capture zone. The sample capture zone is formed on or in a porous material within the sample flow path. Such porous material is also referred to herein generally as an analyte-binding material. It is preferably in the form of a membrane (e.g., a multi-layered material). Such porous material (preferably membrane) allows for fluid flow through the material. This fluid flow can also result in mixing of sample and reagents if desired, within the immunochromatographic device.

The analyte-binding material can include a solid support material having one or more antibodies disposed on the solid support. In certain embodiments, each particle of the particulate material has at least two antibodies that bind different analytes disposed thereon. For example, in certain embodiments, the analyte-binding material includes a solid support material (preferably particulate material) having antibodies MAb-76 and affinity-purified RxClf40 disposed thereon (for example, in a ratio of 1:1, 2:1, 1:2, 3:1, or 1:3) (the recognition element), and a detectable marker.

Suitable methods of attaching antibodies to the solid support include, for example, covalent attachment methods such as disclosed in U.S. Pat. App. Pub. No. 2003/0162236 using cyanoborohydride chemistry in skim milk. Other covalent attachment methods are also known and can be used, as well as non-covalent attachment methods (e.g., passive absorption or adsorption).

The analyte-binding material can include various detectable markers suitable for the detection system desired. For example, such detectable markers (i.e., reporter or detectable moieties or labels) can include a fluorescent label (e.g., such as that described in U.S. Pat. App. Pub. No. 2003/0162236, which discloses fluorescent dye intercalated latex particles). Other detectable markers can include luminescent labels, magnetic labels, chromogenic labels, Raman active labels, and the like.

In certain embodiment of the invention, the various reagents as discussed herein (e.g., lysing agents, mucolytic agents, labeling agents) can be disposed in dry form in a container (e.g., cap, vial, or pipette tip). Such reagents can be dried down using various techniques, such as vacuum drying, and equipment, such as a convection oven and lyophilization. The use of convection oven drying is preferred in certain situations, as much less volume is typically needed to provide the desired amount of the resultant dried reagent.

For drying down reagents, a drying diluent can be used, if desired. An exemplary drying diluent can include, for example, a phosphate buffer, a disaccharide (e.g., trehalose, sucrose), optionally a polysaccharide (e.g., glycerol) specific to conjugation, and a preservative (e.g., sodium azide). Glycerol (i.e., glycerin) is preferably not used if faster drying is desired and/or if convection oven drying is used. A phosphate buffer is preferably present in an amount of at least 5 millimolar (mM), and more preferably at least 10 mM. It is preferably present in an amount of no greater than 500 mM, and more preferably no greater than 50 mM. A disaccharide is preferably present in an amount of at least 0.1 weight percent (wt-%), and more preferably at least 0.5 wt-%. A disaccharide is preferably present in an amount of no greater than 5 wt-%, more preferably no greater than 2 wt-%, and even more preferably no greater than 1 wt-%. A polysaccharide, if used, is preferably present in an amount of at least 1 wt-%. A polysaccharide, if used, is preferably present in an amount of no greater than 20 wt-%, and more preferably no greater than 10 wt-%. A preservative is preferably present in an amount of at least 0.01 wt-%, and more preferably at least 0.2 wt-%. It is preferably present in an amount of no greater than 0.8 wt-%, more preferably no greater than 0.5 wt-%, and even more preferably no greater than 0.1 wt-%.

The use of vials and caps for such vials having reagents therein (particularly, dried-down reagents therein in solid or semi-solid form) can provide greater efficiency, less sample contamination, less sample loss through transfer, better stability, longer shelf life, etc. The vials can be any shape container, and typically are in the shape of tubes.

In certain embodiments, the sample preparation can be done using a sample preparation system that includes: at least one vial and at least one cap for such vial, wherein the vial and the cap each has a different sample preparation reagent disposed therein for the sequential treatment of a sample for analysis of a target analyte; optionally, at least one sample transfer device; and optionally, at least one other sample preparation reagent not in the cap or vial.

In certain alternative embodiments, the sample preparation can be done using a sample preparation system that includes: at least one vial and two or more caps for such vial, wherein each of the caps has a different sample preparation reagent (e.g., an enzymatic lysing agent, a mucolytic agent) disposed therein for the sequential treatment of a sample for analysis of a target analyte; optionally, at least one sample transfer device; and optionally, at least one other sample preparation reagent not in the caps.

Such sample preparation systems could be used in the following exemplary manner: placing a sample into a vial, wherein the vial includes a first reagent (e.g., an enzymatic lysing agent), and maintaining contact between the sample and first reagent under conditions sufficient for reaction between one or more components of the sample and the first reagent (e.g., for a time sufficient to allow lysis of the microorganism and release of at least some antigenic components of the microorganism); and subsequently placing a cap on the vial, wherein the cap includes a second reagent (e.g., mucolytic agent), preferably spotted and dried near the cap's edge, inverting the vial, and mixing the contents of the vial and the second reagent under conditions sufficient for reaction between one or more components in the vial and the second reagent.

The first reagent in the vial, the second reagent in the cap, or both (before reaction) can be in solid or semi-solid form (e.g., in a dried-down coated or spotted form). In one embodiment, the cap referenced above can be a first cap, and after the step of placing the first cap on the vial, the method can further include removing the first cap, replacing it with a second cap that includes a third reagent (e.g., a surfactant), and mixing the contents of the vial and the third reagent under conditions sufficient for reaction between one or more components in the vial and the third reagent.

Another option is where there is no reagent in the vial and that all reagents are added by the addition of caps containing reagents. In a preferred embodiment, the method can further include adding a diluent (e.g., of the type described above) to the contents of the vial after removing the first cap and replacing it with the second cap.

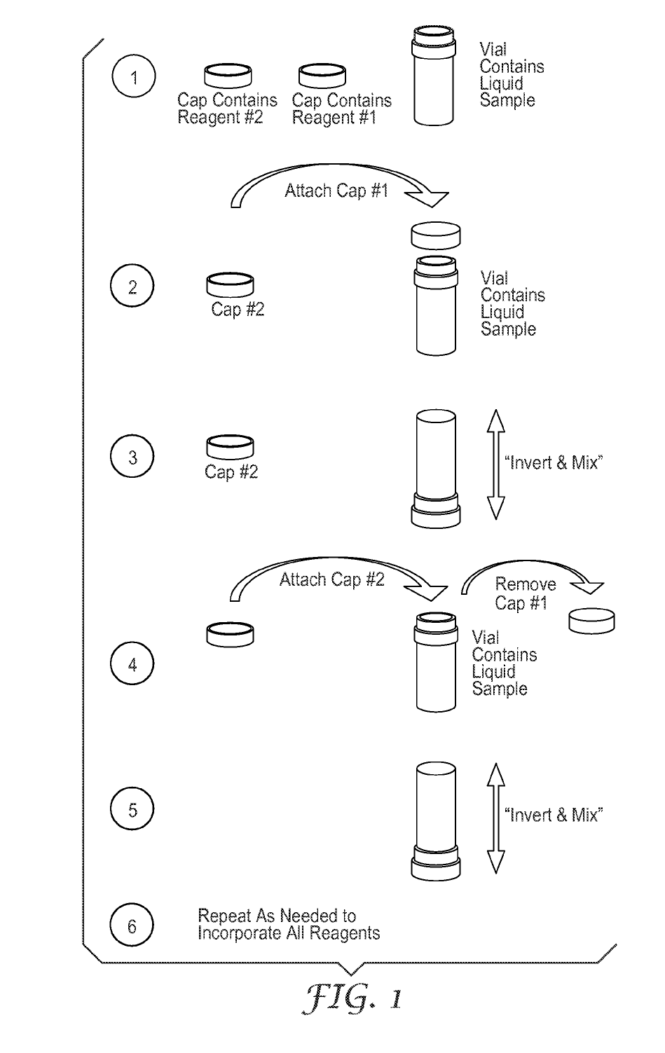

An exemplary embodiment of a sample preparation system and method of the present invention is schematically shown in FIG. 1. A preferred system and method includes a lysing agent (e.g., lystostaphin) in the vial, a mucolytic agent and surfactant (e.g., mixture of n-acetyl cysteine and SDS) in cap 1, and antibody-coated latex particles in cap 2. The various combinations of vials and caps described herein are exemplary, and not necessarily limiting to the systems and methods of the present invention.

As a result of the system and methods of pretreatment (e.g., use of vials and caps for such vials with reagents therein, lysing the cells to form cell-wall fragments, treatment of mucus-containing samples to reduce the interference resulting from the presence of mucus) samples having relatively low concentrations of the species of interest can be evaluated. Thus, advantageously, methods of the invention have improved sensitivity. For example, for certain embodiments, the test sample may include a relatively low concentration of microbes, particularly Staphylococcus aureus. Such relatively low concentrations include, for example, less than 5×10⁴ colony forming units (“cfu”) per milliliter (cfu/mL) of microbe, less than 5×10³ cfu/mL, less than 1000 cfu/mL, and even as low as 500 cfu/mL. Microbes, such as S. aureus, can be detected at high levels as well, ranging up to as much as 5×10⁷ cfu/mL, for example.

Depending on the techniques of analyzing used in the methods of the present invention, relatively small volumes of test sample can be used. Although test sample volume as high as 2 milliliters (mL) may be utilized, advantageously test samples on the order of 10 microliters (μL) are sufficient for certain methods, with 50-100 microliters being preferred for certain embodiments.

Depending on the techniques of analyzing used in the methods of the present invention, the detection time can be relatively short. For example, the detection time can be less than 300 minutes, less than 250 minutes, less than 200 minutes, less than 150 minutes, less than 100 minutes, less than 60 minutes, and even as short as 20 minutes.

Techniques of analyzing useful in methods of the present invention include the use of fluorimetric immunochromatography (e.g., rapid analyte measurement procedure such as that described in U.S. Pat. No. 5,753,517, U.S. Pat. No. 6,509,196, and U.S. Pat. App. Pub. Nos. 2003/0162236 and 2003/0199004).

Preferably, analysis of the analytes in methods of the present invention includes the use of an immunochromatographic device, and preferably a lateral flow device. For example, in preferred embodiments, after sample preparation, methods of the present invention include: providing an analyte-binding material (preferably particulate material) that include two or more antibodies having antigenic specificities for two or more distinct analytes (e.g., separate molecules like protein A and Clumping Factor or two different epitopes of the same molecule) characteristic of the specific microorganism (preferably, a bacterium); providing an immunochromatographic device (preferably, a lateral flow device) that includes a sample capture zone (preferably, in or on a membrane), wherein the sample capture zone includes a mixture of two or more antibodies having antigenic specificities for two or more distinct analytes characteristic of the specific microorganism; providing contact between the sample, the analyte-binding material, and the sample capture zone of the immunochromatographic device; and analyzing for the presence or absence of the specific microorganism. In such methods, for each of the analytes present, at least one of the antigenic specificities of antibodies in the sample capture zone is not functionally blocked from binding to its analyte by the analyte-binding material, having a different antigenic specificity, with analyte bound thereto.

In one particular embodiment, providing contact between the sample, the analyte-binding material, and the sample capture zone of the immunochromatographic device can include: contacting the sample with the analyte-binding material (this contact may occur, for example, within a contact region on the device or before the sample enters the device); and contacting the sample having analyte-binding material therein with the sample capture zone of the immunochromatographic device.

In another particular embodiment, providing contact between the sample, the analyte-binding material, and the sample capture zone of the immunochromatographic device can include: placing the sample into the immunochromatographic device; and subsequently contacting the sample in the device with the analyte-binding material. The immunochromatographic device can be a lateral flow device that includes a sample capture zone in or on a membrane (preferably, nitrocellulose membrane attached to Mylar backing) having antibodies (e.g., MAb 107 and affinity-purified RxClf40) disposed within the sample capture zone, analogous to those devices described in U.S. Pat. App. Pub. No. 2003/0199004. Preferably, the nitrocellulose membrane is blocked against non-specific binding using polyvinyl alcohol.

Optionally, the immunochromatographic device can further include a control capture zone, analogous to those devices described in U.S. Pat. App. Pub. No. 2003/0199004. In one embodiment, affinity-purified GxClf40 can be used in the control capture zone.

Analyzing for the presence or absence of the specific microorganism (preferably, a bacterium) can result from detecting the presence of one or more analytes or the absence of all analytes. Such analysis can occur fluorimetrically.

Significantly, using methods and devices of the present invention, improved sensitivity (i.e., lower levels of detection) and specificity can be realized relative to conventional detection methods for microorganisms, particularly bacteria such as S. aureus. For example, clinical sensitivities of 87% can be realized (i.e., 87% of the S. aureus carriers who had greater than or equal to 5000 cfu/swab were positive using a method and device of the present invention). For example, clinical specificity of 92% can be realized (i.e., 92% of the non-carrier specimens were negative for S. aureus).

### EXAMPLES

The present invention has now been described with reference to several specific embodiments foreseen by the inventor for which enabling descriptions are available. Insubstantial modifications of the invention, including modifications not presently foreseen, may nonetheless constitute equivalents thereto. Thus, the scope of the present invention should not be limited by the details and structures described herein, but rather solely by the following claims, and equivalents thereto.

### Example 1

### Preparation of Sample-Processing Reagent Caps and Tubes

Screw-capped tubes (0.5 mL Screw Cap, MICROT, Sarstedt, Inc.) can be obtained from VWR, Inc. (West Chester, Pa.). The caps are separated from the tubes prior to the addition of the reagents. With the exception of the conjugated latex particles, all reagents are prepared in 0.01M sodium phosphate buffer (pH, 7.2±0.05) containing 0.003M sodium azide, to the final concentrations listed in Table 1. Lysostaphin, trehalose, and N-acetyl cysteine can be obtained from Sigma-Aldrich (St. Louis, Mo.). Sodium dodecylsulfate can be obtained from BIO RAD, Hercules, Calif.

Solutions of lysostaphin and NAC/SDS are prepared as shown in Table 1. A BioDot dispensing machine (Model number AD3200, BioDot, Irvine, Calif.) or the Gilson 402 Diluter Dispenser (Mandel, GF-F410515, Guelph, Ontario, Canada) can be used to spot the solutions into the center of the vials or on the periphery inside the caps. The ingredient concentrations listed in Table 1 are the final concentrations of the solutions that are spotted and dried in the respective reagent containers. The caps and tubes can be dried in a convection oven (VRC2-19, Despatch, Minneapolis, Minn.) at 40° C. for the length of time listed in Table 1. Alternatively, the caps and tubes can be dried in vacuum oven at 20° C., vacuum set point 0 mTorr using a Dura-Stop MP Tray Dryer lyophilizer (FTS Systems, Stone Ridge, N.Y.).

TABLE 1
Ingredients disposed in the caps and tubes for the SA-RAMP assay.

| | | Ingredients | | Drying |
|---|---|---|---|---|
| Reagent | | Final Concentration | Vol. | Time |
| Container | Reagent | (mg/mL) | (ML) | (min) |
| Tube | Bacterio- | Lysostaphin (1.0) | 5 | 150 |
| | lytic | Trehalose (4.18) | | |
| Cap | Mucolytic | N-acetylcysteine (80.0) | 15 | 240 |
| | | Sodium Dodecylsulfate | | |
| | | (13.33) | | |
| | | Trehalose (16.67) | | |
| Cap | Detection | Antibody-conjugated | 5 | 90 |
| | | particles 0.057% | | |
| | | Conjugated particles | | |
| | | Trehalose 5% | | |
| | | Glycerol 1% | | |

### Example 2

### Use of Caps and Tubes in a Method to Prepare a Nasal Sample for Immunochromatographic Analysis

The sample-processing reagent caps and vials from Example 1 can be used in this Example. Swab devices for sample collection can be obtained from Medical Packaging, Inc. (Camarillo, Calif.). The swab device comprises a valve device, capable of delivering a sample elution buffer (0.010M sodium phosphate, 0.15M sodium chloride, pH 8.4-8.6) from a reservoir connected to the swab tube (as described in U.S. Pat. No. 5,879,635). The bulb of the tube is injection-molded using VORIDIAN polyethylene resin (Eastman Chemical Company, Kingsport, Tenn.). The valve is made from Pro-fax polypropylene homopolymer (Basell Polyolefins, Elkton, Md.). The swab consists of a polystyrene hollow barrel of the tube, with spun, adhesive-bound rayon fibers (Kelheim Fibres, Kelheim, Del.). The sheath in which the swab is housed can be formed from extruded polypropylene (Amoco). The entire swab device can be sterilized using an E-beam process with a dose exposure range of 20-25 kGy with and exposure target of 20 kGy. After the sample elution buffer is sterilized in the tube and is urged through the polystyrene hollow barrel of the swab and into the sheath, the pH of the solution is typically 7-8.

Anterior nares samples are collected from one or both nares by firm rotation of a swab device 1-4 times by pressing the swab bud against the septal epithelium to a depth of less than 2.5 centimeters. After collection of the sample, the swab is reinserted into its carrying tube and 0.65 milliliter of PBS sample elution buffer (0.01M sodium phosphate (pH 7.0-8.0)/0.15M sodium chloride) is flushed through the swab bud and the device is vigorously vortexed for 30 seconds to release the sample from the swab. The sample is transferred to a tube containing dried-down lysostaphin reagent and is held at room temperature for 5 minutes.

The screwcap containing the mucolytic reagents is secured on the sample tube and the tube is inverted and vortexed for 30 seconds. The tube is placed right-side up and the bottom of the tube is tapped on a hard surface to move the liquid contents to the bottom of the tube. The screwcap is discarded. The liquid sample is neutralized by adding 4 drops (80-120 mL) of one of the neutralizing buffers described above and disclosed in U.S. Pat. App. Pub. No. 2003/0199004. The cap containing the dried-down antibody-conjugated fluorescent latex particles is secured to the tube. The tube is inverted and vortexed for 30 seconds to resuspend the particles in the sample solution. Within 30 seconds after mixing the liquid sample with the latex particles, 100 mL of the resulting suspension is analyzed using a lateral flow immunochromatographic assay similar to the assay described in International Pat. Pub. No. WO 03/087822 A2.

The complete disclosures of all patents, patent applications, publications, and nucleic acid and protein database entries, including for example GenBank accession numbers, which are cited herein, are hereby incorporated by reference as if individually incorporated. Various modifications and alterations of this invention will become apparent to those skilled in the art without departing from the scope and spirit of this invention, and it should be understood that this invention is not to be unduly limited to the illustrative embodiments set forth herein.

1. A method of analyzing a sample for a bacterium, the method comprising:
providing a sample suspected of including one or more analytes characteristic of a specific bacterium;
providing an analyte-binding material comprising two or more antibodies having antigenic specificities for two or more distinct analytes characteristic of the specific bacterium;
providing an immunochromatographic device comprising a sample capture zone, wherein the sample capture zone comprises a mixture of two or more antibodies having antigenic specificities for two or more distinct analytes characteristic of the specific bacterium;
providing contact between the sample, the analyte-binding material, and the sample capture zone of the immunochromatographic device;
wherein, for each of the analytes present, at least one of the antigenic specificities of antibodies in the sample capture zone is not functionally blocked from binding to its analyte by the analyte-binding material, having a different antigenic specificity, with analyte bound thereto; and
analyzing for the presence or absence of the specific bacterium.

2. The method of claim 1, wherein providing contact between the sample, the analyte-binding material, and the sample capture zone of the immunochromatographic device comprises:
contacting the sample with the analyte-binding material; and
contacting the sample having analyte-binding material therein with the sample capture zone of the immunochromatographic device.

3. The method of claim 1, wherein providing contact between the sample, the analyte-binding material, and the sample capture zone of the immunochromatographic device comprises:
placing the sample into the immunochromatographic device; and
subsequently contacting the sample in the device with the analyte-binding material.

4. The method of claim 1, wherein the antibodies are monoclonal, polyclonal, or combinations thereof.

5. The method of claim 4, wherein the antibodies are selected from the group consisting of MAb-76, MAb-107, affinity-purified RxClf40, affinity-purified GxClf40, fragments thereof, and combinations thereof.

6. The method of claim 5, wherein the analyte-binding material comprises:
a solid support material;
antibodies MAb-76 and affinity-purified RxClf40 disposed on the solid support; and
a detectable marker.

7. The method of claim 5, wherein the sample capture zone comprises antibodies MAb-107 and affinity-purified RxClf40.

8-11. (canceled)

12. The method of claim 1, wherein the sample comprises a mucus-containing sample.

13. (canceled)

14. The method of claim 1, wherein providing a sample comprises:
placing a sample into a vial, wherein the vial includes a first reagent, and maintaining contact between the sample and first reagent under conditions sufficient for reaction between one or more components of the sample and the first reagent; and
subsequently placing a cap on the vial, wherein the cap includes a second reagent, and mixing the contents of the vial and the second reagent under conditions sufficient for reaction between one or more components in the vial and the second reagent.

15. The method of claim 14, wherein the first reagent in the vial, the second reagent in the cap, or both, before reaction is in solid or semi-solid form.

16. The method of claim 14, wherein the cap is a first cap, and after the step of placing the first cap on the vial, the method further comprises removing the first cap, replacing it with a second cap that includes a third reagent, and mixing the contents of the vial and the third reagent under conditions sufficient for reaction between one or more components in the vial and the third reagent.

17-19. (canceled)

20. The method of claim 14, wherein at least one of the reagents comprises a mucolytic agent.

21. The method of claim 20, wherein at least one of the reagents comprises an analyte-binding material, which is added after the mucolytic agent.

22. (canceled)

23. The method of claim 21, wherein the analyte-binding material comprises:
a solid support material;
an antibody disposed on the solid support, wherein the antibody is selected from the group consisting of MAb-76, MAb-107, affinity-purified RxClf40, and combinations thereof; and
a detectable marker.

24. The method of claim 1, wherein providing a sample comprises:
contacting a mucus-containing sample with a first reagent under conditions sufficient for reaction between one or more components of the mucus-containing sample and the first reagent to form a composition, wherein the first reagent comprises an acidified reducing agent having a pH of less than 3;
inactivating the reducing agent in the composition; and
neutralizing the pH of the composition to a pH of 7 to 7.5.

25. The method of claim 24, wherein the inactivating and neutralizing steps occur substantially simultaneously.

26. The method of claim 24, wherein the inactivating and neutralizing steps occur sequentially.

27-29. (canceled)

30. An immunochromatographic device comprising:
a sample flow path; and
a sample capture zone formed on or in a porous material within the sample flow path, the sample capture zone comprising an antibody selected from the group consisting of MAb-76, MAb-107, affinity-purified RxClf40, and combinations thereof.

31. The immunochromatographic device of claim 30, wherein the porous material comprises a membrane.

32. (canceled)

33. The immunochromatographic device of claim 30, wherein the sample capture zone comprises at least two antibodies that bind to different analytes.

34. A system comprising:
an immunochromatographic device comprising:
a sample flow path; and
a sample capture zone formed on or in a porous material within the sample flow path, the sample capture zone comprising one or more antibodies; and
particulate material comprising one or more antibodies disposed thereon;
wherein the one or more antibodies of the sample capture zone, or disposed on the particulate material, or both are selected from the group consisting of MAb-76, MAb-107, affinity-purified RxClf40, and combinations thereof.

35. The system of claim 34, wherein each particle of the particulate material has at least two antibodies that bind different analytes disposed thereon.

36. (canceled)

37. The system of claim 34, wherein the sample capture zone comprises at least two antibodies that bind different analytes.

38. The system of claim 34, wherein the particulate material comprises antibodies MAb-76 and affinity-purified RxClf40 in a 1:1 ratio.

39.-40. (canceled)

41. A method of preparing a mucosal test sample, comprising the steps of:
combining a mucosal sample suspected of containing a microorganism with an enzymatic lysing agent; and
subsequently combining the sample and enzymatic lysing agent with a mucolytic agent that is distinct from the enzymatic lysing agent, thereby forming the mucosal test sample.

42. The method of claim 41, wherein the mucosal sample and enzymatic lysing agent are incubated for a time sufficient to allow lysis of the microorganism and release of at least some antigenic components of the microorganism.

43. The method of claim 41, wherein the enzymatic lysing agent is at least one member selected from the group consisting of lysostaphin, pepsin, glucosidase, galactosidase, lysozyme, achromopeptidase, endopeptidase, N-acetylmuranyl-L-alanine amidase, endo-beta-N-acethylglucosaminidase, ALE-1, and combinations thereof.

44. (canceled)

45. The method of claim 41, wherein the mucolytic agent is at least one member selected from the group consisting of an enzyme, a salt, a reducing agent, an acid, and combinations thereof.

46. The method of claim 45, wherein the mucolytic agent is a reducing agent.

47. The method of claim 46, wherein the reducing agent is at least one member selected from the group consisting of beta-mercapto ethanol, dithiotreotol, dithioerythritol, cysteine, tris(2-carboxyethyl) phosphine hydrochloride, n-acetyl cysteine, and combinations thereof.

48. The method of claim 47, wherein the reducing agent includes n-acetyl cysteine.

49. The method of claim 48, wherein the reducing agent has a pH of less than 3.

50. The method of claim 49, further including the step of adjusting the pH of the reducing agent with an acid to obtain a pH of less than 3.

51. The method of claim 41, further including a step of combining the mucosal test sample with a surfactant subsequently to, or concurrently with, combining the sample and enzymatic lysing agent with a mucolytic agent.

52.-53. (canceled)

54. The method of claim 51, further including a step of combining the mucosal test sample and surfactant with a neutralizing buffer.

55. The method of claim 54, wherein the neutralizing buffer is sufficient to adjust the pH of the mucolytic test sample and surfactant to a range of 5 to 8.

56. (canceled)

57. The method of claim 41, further including the step of combining the mucosal test sample with an analyte recognition element, whereby the presence of one or more analytes characteristic of the microorganism can be detected.

58.-59. (canceled)

60. The method of claim 57, wherein the analyte recognition element includes at least two antibodies, whereby the antibodies bind, independently, distinct antigenic components of Staphylococcus aureus produced by lysis, or bind, independently, distinct epitopes of a common antigenic component produced by the lysis.

61. The method of claim 60, wherein the antibodies are selected from the group consisting of MAb-76, MAb-107, RXCLF 40, and combinations thereof.

62. (canceled)

63. A method for preparing a nasal mucosal test sample, comprising the steps of
combining a nasal mucosal sample of a Staphylococcus aureus with lysostaphin;
subsequently combining the nasal mucosal sample and lysostaphin with n-acetyl cysteine having a pH below 3 and with sodium dodecyl sulfate;
inactivating the combined nasal mucosal sample, lysostaphin, n-acetyl cysteine and sodium dodecyl sulfate by neutralizing the pH to a range of 5 to 8 to thereby form a neutralized test sample; and
combining the neutralized test sample with a labeled recognition element comprising at least two antibodies selected from the group consisting of MAb-76, MAb-107, RXCLF 40, and combinations thereof.

64. The method of claim 63, wherein the nasal mucosal sample and lysostaphin are incubated for a time sufficient to allow lysis of the Staphylococcus aureus and release of at least some of its antigenic components.

65. A sample preparation system comprising:
at least one vial and at least one cap for such vial, wherein the vial and the cap each has a different sample preparation reagent disposed therein for the sequential treatment of a sample for analysis of a target analyte;
optionally, at least one sample transfer device; and
optionally, at least one other sample preparation reagent not in the cap or vial.

66. A sample preparation system comprising:
at least one vial and two or more caps for such vial, wherein each of the caps has a different sample preparation reagent disposed therein for the sequential treatment of a sample for analysis of a target analyte;
optionally, at least one sample transfer device; and
optionally, at least one other sample preparation reagent not in the caps.

67.-70. (canceled)
